(19) United States
(12) Patent Application Publication
(10) Pub. No.: US 2006/0264969 A1
(43) Pub. Date: Nov. 23, 2006

(54) Method and device for determination of the mobility of a hip joint prosthesis

(75) Inventors: Francois Leitner, Uriage (FR); Benoit Mollard, Eybens (FR)

Correspondence Address: Lipsitz & McAllister, LLC, 755 MAIN STREET, MONROE, CT, 06468, US

(73) Assignee: AESCULAP AG & CO. KG, Tuttlingen (DE)

(21) Appl. No.: 11/389,521

(22) Filed: Mar. 23, 2006

Related U.S. Application Data
(63) Continuation of application No. PCT/EP03/10779, filed on Sep. 27, 2003.

(51) Int. Cl. A61B 17/56

(52) U.S. Cl. 606/102; 623/914

(57) ABSTRACT

A method for determination of the mobility of a hip joint prosthesis is proposed, comprising a bearing cup that can be inserted in the hip bone and a prosthetic shaft that can be inserted in the femur and is pivotally mounted by a spherical joint surface in the bearing cup, which is characterised in that the femur with the prosthetic shaft is pivoted as far as possible in various directions, in that it is determined whether the central point of the bearing cup and the central point of the spherical joint surface move apart from one another during this process, and in that, for each pivoting direction, the maximum pivoting angle is thus determined, at which the central points begin to move apart from one another. A device for carrying out this method is also proposed.

This application is a continuation of international application number PCT/EP03/10779 filed on Sep. 27, 2003.

The present disclosure relates to the subject matter disclosed in international application PCT/EP03/10779 of Sep. 27, 2003, which is incorporated herein by reference in its entirety and for all purposes.

### BACKGROUND OF THE INVENTION

The invention relates to a method for determination of the mobility of a hip joint prosthesis comprising a bearing cup that can be inserted in the hip bone and a prosthetic shaft that can be inserted in the femur and is pivotally mounted by a spherical joint surface in the bearing cup.

The invention also relates to a device for carrying out this method.

During insertion of hip joint prostheses, a bearing cup is generally inserted in the hip joint and a prosthetic shaft is inserted in the femur, this prosthetic shaft engages with a spherical joint surface in the bearing cup, which is also spherical, so the central points of the bearing cup and the spherical joint surface of the prosthetic shaft coincide. Owing to this mounting, the femur can be pivoted in all directions with respect to the hip bone, this pivoting movement is limited, on the one hand, by the surrounding soft tissue and, on the other hand, this pivoting movement can be limited in that the bony structures of the hip joint and femur strike against one another or against parts of the prosthesis. This limitation of the pivoting movement may be undesired, and it is therefore necessary to establish during the operation whether the desired mobility of the femur with respect to the hip bone is achieved after insertion of the hip joint prosthesis.

The invention is accordingly based on the object of providing a method for determination of the mobility of a hip joint prosthesis which makes it possible for the operator to determine the pivoting range available.

### SUMMARY OF THE INVENTION

This object is achieved according to the invention in a method of the type described at the outset in that the femur with the prosthetic shaft is pivoted as far as possible in various directions, in that it is determined whether the central point of the bearing cup and the central point of the spherical joint surface move apart from one another during this process and in that, for each pivoting direction, the maximum pivoting angle is determined, at which the central points begin to move apart from one another.

When the pivoting movement of the femur is limited by one of said stops, in other words by direct contact of bony structures, by contact of prosthetic parts with bony structures or by contact of prosthetic parts with one another, a further pivoting of the femur with respect to the hip bone can then only be achieved in that pivoting takes place about the contact point, and this inevitably leads to the spherical joint surface being lifted from the bearing cup, i.e. the spacing increases between the central points of the bearing cup, on the one hand, and of the spherical joint surface, on the other hand, which points coincide with normal fitting of the spherical joint surface in the bearing cup. Thus, if it is determined that these central points move apart from one another, in other words a luxation occurs in the hip joint, this is an indication that a stop is preventing further pivoting movement of the femur with respect to the hip joint.

In order to be able to determine this moving apart of the central points, it is favourable if the positional data both of the bearing cup and also of the prosthetic shaft is determined with the aid of a navigation system. These types of navigation systems are conventional in the case of surgical operations; these are systems which are able to determine the position of so-called marking elements in the space, for example by emitting and receiving infrared radiation, which is reflected by these marking elements. These marking elements are rigidly fixed on the parts, the position of which is to be determined, so their position in the space is also determined in this manner by the navigation system.

It may be provided, in particular, that marking elements of the navigation system are placed on the hip bone and the femur. The positions of the hip bone, on the one hand, and the femur, on the other hand, are monitored by these marking elements, the navigation system determining in all cases both the position and the orientation in the space; the total of this data is called the “position” below.

It is particularly advantageous if the position of the central points of the bearing cup and the spherical joint surface is determined by pivoting the femur with respect to the hip bone by an angle which is below the maximum pivoting angle. During pivoting of this type, the two central points coincide, and the marking elements move about these central points on a bearing cup, so the central points have to be determined. The precise position of the central points both of the bearing cup and the spherical joint surface is thus obtained by determining the position of the femur and the hip bone and on pivoting the hip bone beyond the maximum pivoting angle, the luxation of the hip joint can be observed, in other words the enlargement of the spacing between the central points of the bearing cup, on the one hand, and the spherical joint surface, on the other hand.

A test shaft, in other words a prosthesis which is only inserted temporarily into the femur and only used for measuring and adjustment purposes, is preferably used as the prosthetic shaft and as soon as the position has been precisely determined, this test shaft is replaced by the actual prosthetic shaft, which has corresponding dimensions and is placed in the femur in the same manner.

In a particularly preferred embodiment, it is provided that a bearing cup is reproduced on a display and the maximum pivoting angle is shown along the periphery thereof. The operator can then immediately read off from this display the maximum pivoting angles, which are produced on pivoting the femur in different pivoting directions.

In particular, the peripheral angle ranges of the bearing cup can be marked in the process, in which the maximum pivoting angel is below a desired value. While the maximum pivoting angles are determined in specific directions by anatomic conditions, it can be recognised in this manner that the maximum achievable pivoting angle is below this anatomic maximum angle in certain circumstances and must be caused by undesired stops. The stops can be removed by the operator if necessary, for example by removing bone material or by changing the position of the bearing cup, possibly also by using a different bearing cup, for example an asymmetrical bearing cup, or by using another prosthetic shaft, which avoids the undesired stop owing to other dimensions.

It is favourable if, after removal of the limitation of the pivoting movement in a specific peripheral angle range, the maximum pivoting angle is determined afresh in this range, in order to be sure that the undesired stop has been removed to such an extent that the desired maximum angle can be achieved.

The invention is also based on the object of providing a device for carrying out this method.

A device of this type is characterised according to the invention in that a navigation system is provided for determining the position of the hip bone and the femur, in that a data processing device is provided which calculates the central points of the bearing cup and the spherical joint surface and their mutual spacing from the positional data of the hip bone and the femur determined in this manner, and which generates a signal when the mutual spacing of the central points exceeds a specific value.

In particular, the data processing device can determine the pivoting direction of the femur relative to the hip bone, on generation of the signal, so the maximum pivoting angle can be displayed to the operator for each pivoting direction.

In a preferred embodiment, a display is provided, on which the data processing device reproduces a bearing cup and shows the maximum pivoting angle along the periphery thereof.

It may be provided in the process that the data processing device marks the peripheral angle ranges of the bearing cup, in which the maximum pivoting angle is below a desired value.

The prosthetic shaft is preferably a test shaft.

The following description of preferred embodiments of the invention is used in conjunction with the drawings for more detailed description.

### BRIEF DESCRIPTION OF THE DRAWINGS

FIG. 1 shows a schematic view of a device for determination of the maximum pivoting angle of a femur with respect to a hip bone;

FIG. 2 shows an enlarged view of the hip joint with a hip joint prosthesis inserted and with marking elements on the femur and hip bone;

FIG. 3a: shows a sectional view through the hip joint in the region A in FIG. 2 with normal mounting of the spherical joint surface of the prosthetic shaft in the bearing cup;

FIG. 3b: shows a view similar to FIG. 3a with the spherical joint surface of the prosthetic shaft lifted out of the bearing cup and

FIG. 4: shows a schematic view of a display of a bearing cup with a marked luxation region.

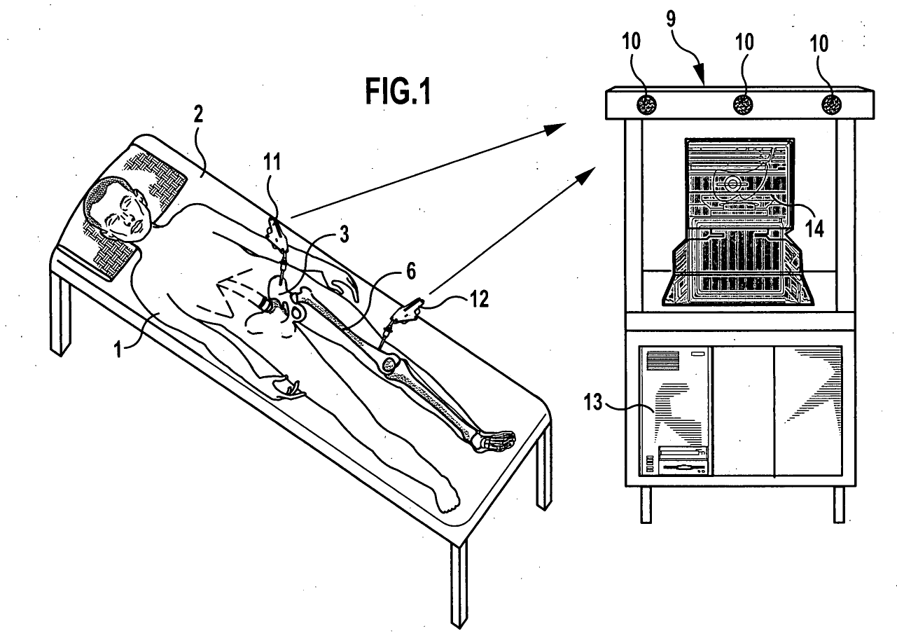

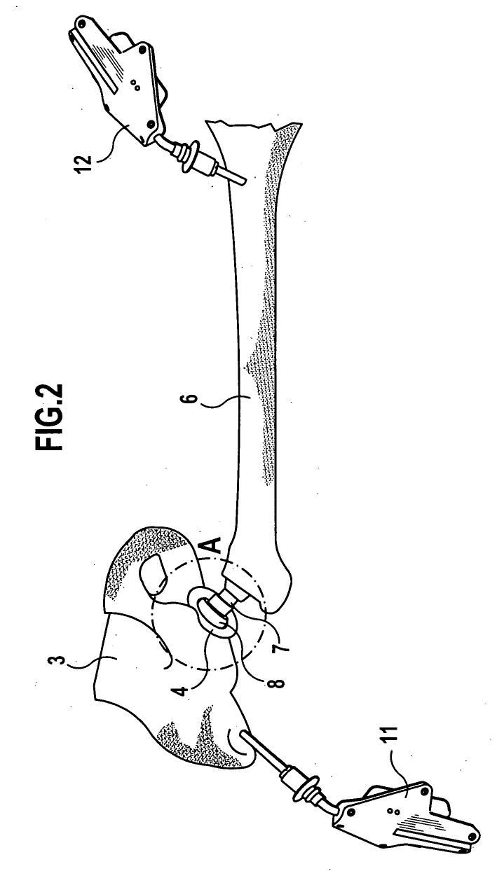

### DETAILED DESCRIPTION OF THE INVENTION

In an operating theatre, a patient 1 lies on an operating table 2, where an artificial hip joint is to be inserted. For this purpose, a bearing cup 4 with a substantially semi-spherical concave bearing surface 5 is inserted in the hip bone 3 and a prosthetic shaft 7 is inserted in the femur 6, the prosthetic shaft carrying a convex, spherical joint surface 8 on its end projecting out of the femur 6. This spherical joint surface 8 has the same diameter as the concave spherical bearing surface 5 and engages in the latter such that the central points of the spherical joint surface 8 and the bearing surface 5 coincide; a ball joint is formed which makes pivoting of the femur 6 possible in all directions with respect to the hip bone 3.

In order to be able to follow these pivoting movements, a navigation system 9 is provided, which continually determines the position, in other words the location and the orientation of the hip bone 3 and femur 6 in the space. The navigation system 9 comprises a plurality of emitting and receiving mechanisms 10 arranged at a spacing from one another and two marking elements 11, 12, which are rigidly connected, for example via bone screws to the hip bone 3 or the femur 6. The marking elements 11, 12 receive, at various locations arranged spaced apart from one another, radiation emitted by the emitting and receiving mechanisms 10, for example infrared radiation, and reflect this onto the emitting and receiving mechanisms 10. The navigation system 9 can determine the position of the marking elements 11, 12 in the space on the basis of this reflected radiation, and therefore the position of the hip bone 3 and femur 6.

A data processing device 13, which receives the positional data determined by the navigation system 9 and processes it further, is associated with the navigation system 9, as is also a screen 14, on which the data processing device 13 shows this data or data calculated therefrom.

The operator will carry out the following steps, preferably using so-called test implants, in other words using a prosthetic shaft 7, which is not yet the final prosthetic shaft, but is only temporarily inserted in the femur as a test, in order to check the kinematics of the joint and the correct implantation position with the aid of this test shaft. The same may apply with regard to the bearing cup 4; it is also possible here to use a test cup. These test implants are removed after successful alignment and positioning and replaced by final implants, which have the same geometry and are implanted in the same way as the test implants.

To determine the mobility of the femur with respect to the hip bone, the positions of the central points of the bearing cup 4, on the one hand, and of the spherical joint surface 8, on the other hand, are initially determined relative to the hip bone 3 or to the femur 6. For this purpose, the femur 6 is pivoted relative to the hip bone 3 in various directions. As the hip joint is a ball joint, the marking elements 11 and 12 move in the process on spherical surfaces, the central point of which is defined by the central point of the ball joint. With normal mounting, the muscles pull the spherical joint surface into the bearing cup 4, so the spherical joint surface 8 rests flat on the bearing surface 5; the central points of the bearing cup 4 and the spherical joint surface 8 therefore coincide. The position of the two central points can thus be determined in the same manner relative to the hip bone 3, on the one hand, and to the femur 6, on the other hand, by the described pivoting movement of the femur.

In a second step, the operator now checks how far the femur can be pivoted in various directions in the hip joint. For this purpose, the femur is pivoted as far as possible relative to the hip bone. In some directions, this pivoting movement is limited by the surrounding muscle tissue; pivoting movements going beyond this are prevented thereby.

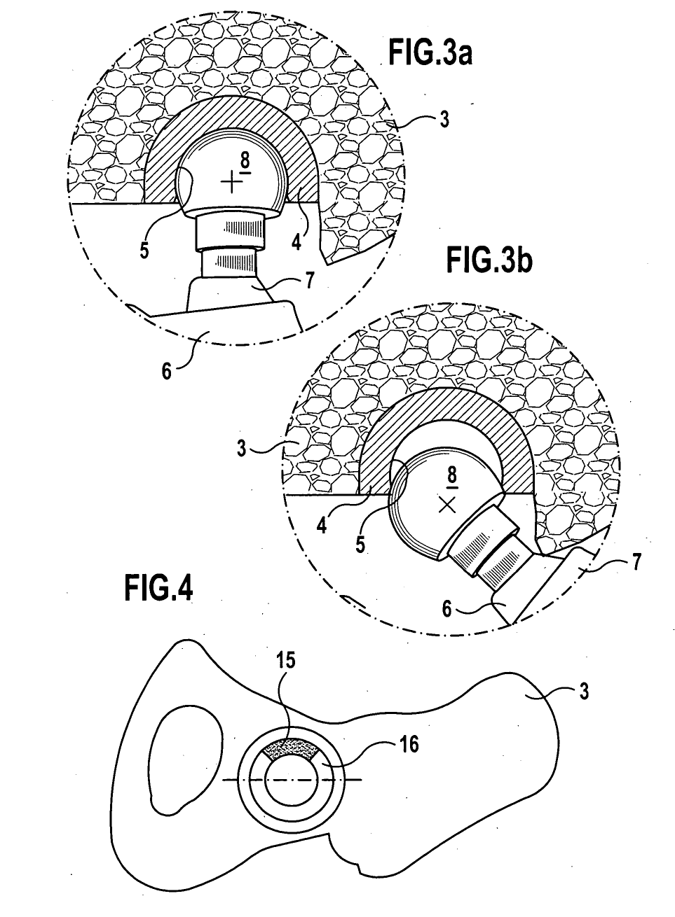

However, in other directions, in which the muscle tissue would allow a further pivoting movement, this pivoting movement can be hindered in that the two parts moved against one another strike against one another. This may be a stop between bony structures of the femur and hip bone, the bony structure of one part may also strike against the prosthetic part of the other part, and the two prosthetic parts, in other words the bearing cup and prosthetic shaft, may also strike against one another. If this is the case and if the operator then continues the pivoting movement of the femur, the stop point forms an abutment, and this leads to the spherical joint surface 8 being lifted out of the bearing cup 4 and a luxation of the hip joint is thus produced, as shown in FIG. 3b. In this process, the central points of the bearing surface 5, on the one hand, and of the spherical joint surface 8, on the other hand, move apart from one another, these having adopted the same position, in other words a spacing of practically zero, prior to the abutting of the two moved parts.

The data processing device continuously receives positional data via the navigation system 9 both for the hip bone 3 and the femur 6 and from this positional data, can calculate the respective positions of the central points of the bearing surface 5 and of the spherical joint surface 8 and therefore also their spacing apart.

As soon as a spacing of this type occurs, the data processing device generates a signal, from which the operator can infer that a luxation is beginning, in other words that a stop must have been produced. The data processing device can also determine from the positional data, in which direction the femur has been pivoted in the process relative to the hip bone; in other words the operator, in addition, also receives information concerning at what peripheral angle of the bearing cup 4 this stop has occurred.

In addition, the operator can be provided with an image of the bearing cup 4 on the screen, for example in the form of a ring, and the peripheral angle range of the bearing cup, in which a stop has been produced, in which, in other words, a luxation has occurred, can be marked in this image. An image of this type is shown schematically in FIG. 4. A region 15 is marked there in the annular image 16 of a bearing cup, and a luxation is produced in this region if the operator pivots the femur into this peripheral region of the bearing cup. The data processing device can also calculate the pivoting angle from the positional data, so the operator can, if necessary, also obtain information as to whether the luxation only occurs when a maximum desired pivoting angle is exceeded—the stop is harmless then, because it only occurs at pivoting angles which are normally not required—or whether the luxation already occurs at a pivoting angle, which is below the maximum desired pivoting angle, in which case a limitation of this type of the pivoting movement is disruptive and therefore has to be eliminated.

For this purpose, the operator has a plurality of possibilities available, for example he can remove bony structures in the stop region. It is also possible to use other prosthetic parts, which have a smaller overall size in this region and therefore avoid a stop. There is also the possibility of using asymmetrical bearing cups, which have reduced geometries in specific angle ranges. After such removal of a stop, the described method is repeated, in order to determine whether, after the removal of the stop, the maximum desired pivoting angle of the femur can be achieved without luxation.

The determination of a luxation, i.e. a distancing of the central points of the bearing surface 5 and spherical joint surface 8 is thus always an indication that the pivoting movement of the femur in a specific direction is limited by a stop, and this information can be used by the operator to check the desired mobility of the hip joint in the described manner.

1. Method for determination of the mobility of a hip joint prosthesis comprising a bearing cup that can be inserted in the hip bone and a prosthetic shaft that can be inserted in the femur and is pivotally mounted by a spherical joint surface in the bearing cup, wherein the femur with the prosthetic shaft is pivoted as far as possible in various directions, it is determined whether the central point of the bearing cup and the central point of the spherical joint surface move apart from one another during this process, and for each pivoting direction, the maximum pivoting angle is thus determined, at which the central points begin to move apart from one another.

2. Method according to claim 1, wherein the positional data both of the bearing cup and also of the prosthetic shaft is determined with the aid of a navigation system.

3. Method according to claim 2, wherein marking elements of the navigation system are placed on the hip joint bone and on the femur.

4. Method according to claim 2, wherein the position of the central points of the bearing cup and the spherical joint surface is determined by pivoting the femur with respect to the hip bone by an angle which is below the maximum pivoting angle.

5. Method according to claim 3, wherein the position of the central points of the bearing cup and the spherical joint surface is determined by pivoting the femur with respect to the hip bone by an angle which is below the maximum pivoting angle.

6. Method according claim 1, wherein a test shaft is used as the prosthetic shaft.

7. Method according to claim 1, wherein a bearing cup is reproduced on a display and the maximum pivoting angle is shown along the periphery thereof.

8. Method according to claim 7, wherein there are marked peripheral angle ranges of the bearing cup, in which the maximum pivoting angle is below a desired value.

9. Method according to claim 1, wherein the limitation of the pivoting movement in a specific peripheral angle range is removed and the maximum pivoting angle is determined afresh in this peripheral angle range.

10. Method for determination of the mobility of a hip joint prosthesis comprising a bearing cup that can be inserted in the hip bone and a prosthetic shaft that can be inserted in the femur and is pivotally mounted by a spherical joint surface in the bearing cup, wherein a navigation system is provided for determining the position of the hip bone and the femur, and a data processing device is provided, which calculates the central points of the bearing cup and the spherical joint surface and their mutual spacing from the determined positional data of the hip bone and the femur and which generates a signal when the mutual spacing of the central points exceeds a specific value.

11. Device according to claim 10, wherein the data processing device, on generation of the signal, determines the pivoting direction of the femur relative to the hip bone.

12. Device according to claim 11, wherein a display is provided on which the data processing device reproduces a bearing cup and shows the maximum pivoting angle along the periphery thereof.

13. Device according to claim 12, wherein the data processing device marks the peripheral angle ranges of the bearing cup, in which the maximum pivoting angle is below a desired value.

14. Device according to claim 10, wherein the prosthetic shaft is a test shaft.

15. Device according to claim 11, wherein the prosthetic shaft is a test shaft.

16. Device according to claim 12, wherein the prosthetic shaft is a test shaft.

17. Device according to claim 13, wherein the prosthetic shaft is a test shaft.
